(19) United States
(12) Patent Application Publication
(10) Pub. No.: US 2026/0217626 A1
(43) Pub. Date: Jul. 30, 2026

(54) PROCESS FOR PRODUCING C2-C4 OLEFINS FROM METHANOL

(71) Applicant: BASF SE, Ludwigshafen am Rhein (DE)

(72) Inventors: Andre BADER, Ludwigshafen am Rhein (DE); Lee Russell COLLINS, Ludwigshafen am Rhein (DE); Andreas JOERKE, Ludwigshafen am Rhein (DE); Paul-Vinzent STROBEL, Ludwigshafen am Rhein (DE); Robert Peter Michael FRANZ, Ludwigshafen am Rhein (DE); Johannes Lazaros Friedrich ELLER, Ludwigshafen am Rhein (DE); Carsten DRECHSLER, Ludwigshafen am Rhein (DE); Sebastian POHL, Ludwigshafen am Rhein (DE)

(21) Appl. No.: 19/148,701

(22) PCT Filed: Jan. 17, 2024
(86) PCT No.: PCT/EP2024/050991

§ 371 Date: Jul. 17, 2025

(30) Foreign Application Priority Data
Jan. 18, 2023 (EP) 23152136.0

(51) Int. Cl. C07C 1/20; C07C 11/04; C07C 11/06; C07C 41/09; C07C 43/04

(52) U.S. Cl. CPC C07C 1/20; C07C 11/04; C07C 11/06; C07C 41/09; C07C 43/043

(57) ABSTRACT

The invention relates to a process for preparing C₂-C₄ olefins from methanol, comprising the steps of:
A) feeding a feed stream A comprising methanol into a dimethyl ether fixed bed reactor and catalytically converting methanol to dimethyl ether to obtain a product stream A1 comprising dimethyl ether, methanol and water vapor;
B) mixing stream A1 with one or more hydrocarbon recycle streams R comprising C₂-C₆ hydrocarbons and catalytically converting it in an olefin fixed bed reactor to a crude product stream B comprising C₂-C₄ olefins, C₅-C₆ hydrocarbon and C₆⁺ hydrocarbons;
C) cooling crude product stream B to obtain a hydrocarbon crude product stream C;
D) separating hydrocarbon crude product stream C into at least one ethylene-comprising stream, at least one propylene-comprising stream, at least one stream comprising butenes, at least one stream comprising C₅-C₆ hydrocarbons, and at least one by-product stream N comprising C₆⁺ hydrocarbons;
E) recycling a portion of the C₂-C₄ olefins selected from ethylene, propylene and butenes that are obtained in step D), and at least a portion of the C₅-C₆ hydrocarbons that are obtained in step D) as one or more hydrocarbon recycle streams R into step B);
F) obtaining at least one ethylene-comprising product-of-value stream F1, at least one propylene-comprising product stream F2 and optionally one or more product-of-value streams F3 comprising butenes from the streams obtained in step D);
G) feeding at least a portion of the propylene-comprising product stream F2 into an olefin metathesis reactor and converting propylene to a product stream G comprising ethylene and butenes, leaving a substream F4 of stream F2 as the product-of-value stream;
H) separating an ethylene-comprising product-of-value stream H1 and a product-of-value stream H3 comprising butenes from the product stream G;
I) optionally recycling at least a portion of the butenes present in product-of-value stream H3 into step B), leaving a substream H4 as product-of-value stream;
K) discharging the at least one by-product stream N comprising C₆⁺ hydrocarbons;
wherein
based on 100% by weight of the C₂-C₄ olefins present as products of value in streams F1, F3, F4, H1 and H3 or H4, 30% to 60% by weight of ethylene, 30% to 60% by weight of propylene and 0% to 30% by weight of butenes are present and are recovered as products of value, and, based on C₂-C₄ olefins present in the one or more recycle streams R and the product-of-value and by-product streams F1, F3, F4, H1, N and H3 or H4, 0% to 10% of the ethylene, 30% to 60% of the propylene and 40% to 80% of the butenes are recycled into step B), and, based on crude product stream B, 20% to 40% of the propylene is conducted into the olefin metathesis reactor, or, based on the C₂-C₄ olefins present in the one or more recycle streams R and the product-of-value and by-product streams F1, F3, F4, H1, N and H3 or H4, 5% to 30% of the ethylene, 0% to 10% of the propylene and 40% to 85% of the butenes are recycled into step B), and, based on crude product stream B, 50% to 70% of the propylene is conducted into the olefin metathesis reactor.

The invention relates to a process for preparing C₂-C₄ olefins from methanol.

It is known that propylene can be produced by converting a methanol/dimethyl ether mixture in a fixed bed reactor (methanol-to-propylene, MTP reactor). Known fixed bed reactors are operated with zeolite catalysts at temperatures of about 480° C.

US 2010/145125 A1 discloses a process for preparing light olefins by the conversion of methanol and ethanol. The process comprises the feeding of a first part-feed via a distributor at the base of a fluidized bed reactor to a reaction zone comprising a catalyst, the feeding of a second part-feed from at least one point above the distributor to the reaction zone, the contacting of the feed with the catalyst and reacting to give a stream comprising ethylene and propylene, where the first and second part-feeds each independently comprise methanol and/or ethanol, with the proviso that the feed overall comprises both methanol and ethanol, and the weight ratio of methanol to ethanol in the feed overall is in the range from 99:1 to 0.1:1.

CN 216106699 U discloses a process for preparing ethylene, propylene and butenes by catalytic dehydration of methanol. The system comprises a reaction unit and a separation unit, wherein the reaction unit comprises a preliminary reactor, a process vapor column and a main reactor, and the separation unit comprises a quencher and a compressor. The main reactor comprises a ZSM-5 molecular sieve catalyst; the separating unit comprises an ethylene column, a propylene column and a butenes column. Ethane, propane, butane and C₅ and C₆ hydrocarbons are partly recycled into the main reactor; further components are discharged as by-products. The unit can also be used to prepare butenes while the amount of recycled hydrocarbons is reduced and the energy consumption of the unit is lowered. Only saturated hydrocarbons are recycled into the main reactor.

CN 110218138 A discloses a process for increasing the yield of olefin in a methanol-to-propylene process (MTP process), in which C₂ hydrocarbons, C₄ hydrocarbons and C₅-C₇ hydrocarbons are recycled into the MTP reactor. In one example, in a 500 000 t/a methanol-to-propylene plant, the methanol feed is 210 t/h, and those of the recycled C₂, C₄ and C₅-C₇ hydrocarbons are 27 t/h, 40 t/h and 150 t/h respectively. Ethylene production is 1.5 t/h; propylene production is 60 t/h.

There is no mention of co-feeding of ethanol. The product of value obtained is very predominantly propylene.

U.S. Pat. No. 11,136,278 B2 discloses a process for converting propylene to ethylene, comprising feeding a propylene feed stream into a C3 metathesis reactor and converting it to ethylene and 2-butene; separating in a fractionation system into an ethylene fraction, a C3 fraction, a C4 fraction and a C5+ fraction; feeding at least a portion of the C3 fraction into the C3 metathesis reactor to produce additional ethylene; feeding the C4 fraction into a C4 isomerization/metathesis reaction zone and converting the C4 fraction by: (i) isomerizing a portion of the 2-butenes to 1-butene, (ii) metathesizing 1-butene and 2-butene to propylene and 2-pentene, and/or (iii) autometathesizing 1-butene to ethylene and 3-hexene; obtaining a product stream comprising ethylene, propylene, butenes, pentenes and hexenes and feeding it into the fractionation system.

U.S. Pat. No. 11,046,627 B2 discloses a process for preparing propylene from ethylene and butylene, comprising:
(a) producing, in a methanol-to-propylene (MTP) system comprising a zeolite catalyst, and a methanol-to-olefin (MTO) system comprising a silicoaluminophosphate (SAPO) molecular sieve catalyst, a first product stream comprising ethylene and a second product stream comprising butylene, where the MTP or MTO system comprises:
(i) a first reactor that converts methanol to dimethyl ether; and
(ii) a second reactor that converts the dimethyl ether to a C2+ product stream comprising propylene, ethylene and butylene;
(b) feeding the first and second product streams to a metathesis reaction unit and converting the ethylene and butylene to a metathesis product stream comprising propylene and unconverted ethylene and butylene;
(c) separating the propylene from unconverted ethylene and butylene to produce a propylene product stream and a stream comprising unconverted ethylene and butylene;
(d) recycling the stream comprising the unconverted ethylene and butylene from step (c) into the MTP or MTO system.

EP 2 892 863 B1 comprises a process for preparing linear butenes from methanol, comprising the steps of: a) providing methanol; b) converting the methanol in a first reaction stage to a first reaction mixture comprising dimethyl ether and water, with or without unconverted methanol; c) converting dimethyl ether in a second reaction stage to a second reaction mixture comprising propene and further hydrocarbons having two, four and five carbon atoms, where the second reaction stage is fed at least partly from the first reaction mixture; d) working up the second reaction mixture to obtain a propene-rich fraction and at least one low-propene fraction, where the low-propene fraction is partially returned to the second reaction stage; e) converting propene in a third reaction stage to a third reaction mixture comprising ethene and linear butenes selected from the group comprising 1-butene, cis-2-butene, trans-2-butene, where the third reaction stage is fed at least partly from the propene-rich fraction; f) working up the third reaction mixture to a target fraction rich in linear butenes and to an ethene-rich fraction.

It is an object of the invention to provide a flexible process for preparing C₂-C₄ olefins from methanol in a methanol-to-olefin process (MTO process), in which the proportions of ethylene, propylene and butenes in the product-of-value streams obtained from the process can be varied within wide ranges, and the volume of the recycle streams recycled into the MTO process can be reduced overall.

The object is achieved by a process for preparing C₂-C₄ olefins from methanol, comprising the steps of:
A) feeding a feed stream A comprising methanol into a dimethyl ether fixed bed reactor and catalytically converting methanol to dimethyl ether to obtain a product stream A1 comprising dimethyl ether, methanol and water vapor;
B) mixing stream A1 with one or more hydrocarbon recycle streams R comprising C₂-C₆ hydrocarbons and catalytically converting it in an olefin fixed bed reactor to a crude product stream B comprising C₂-C₄ olefins, C₅-C₆ hydrocarbon and C₆⁺ hydrocarbons;
C) cooling crude product stream B to obtain a hydrocarbon crude product stream C;
D) separating hydrocarbon crude product stream C into at least one ethylene-comprising stream, at least one propylene-comprising stream, at least one stream comprising butenes, at least one stream comprising C₅-C₆ hydrocarbons, and at least one by-product stream N comprising C₆⁺ hydrocarbons;
E) recycling a portion of the C₂-C₄ olefins selected from ethylene, propylene and butenes that are obtained in step D), and at least a portion of the C₅-C₆ hydrocarbons that are obtained in step D) as one or more hydrocarbon recycle streams R into step B);
F) obtaining at least one ethylene-comprising product-of-value stream F1, at least one propylene-comprising product stream F2 and optionally one or more product-of-value streams F3 comprising butenes from the streams obtained in step D);
G) feeding a portion of the propylene-comprising product stream F2 into an olefin metathesis reactor and converting propylene to a product stream G comprising ethylene and butenes, leaving a substream F4 of stream F2 as the product-of-value stream;
H) separating an ethylene-comprising product-of-value stream H1 and a product-of-value stream H3 comprising butenes from the product stream G;
I) optionally recycling a portion of the butenes present in product-of-value stream H3 into step B), leaving a stream H4 as product-of-value stream;
K) discharging the at least one by-product stream N comprising C₆⁺ hydrocarbons;
   wherein
   based on 100% by weight of the C₂-C₄ olefins present as products of value in streams F1, F3, F4, H1 and H3 or H4, 30% to 60% by weight of ethylene, 30% to 60% by weight of propylene and 0% to 30% by weight of butenes are present and are recovered as products of value, and, based on the C₂-C₄ olefins present in the one or more recycle streams R and the product-of-value and by-product streams F1, F3, F4, H1, N and H3 or H4, 0% to 10% of the ethylene, 30% to 60% of the propylene and 40% to 80% of the butenes are recycled into step B), and, based on crude product stream B, 20% to 40% of the propylene is conducted into the olefin metathesis reactor, or, based on the C₂-C₄ olefins present in the one or more recycle streams R and the product-of-value and by-product streams F1, F3, F4, H1, N and H3 or H4, 5% to 30% of the ethylene, 0% to 10% of the propylene and 40% to 85% of the butenes are recycled into step B), and, based on crude product stream B, 50% to 70% of the propylene is conducted into the olefin metathesis reactor.

The olefin metathesis allows a non-ideal olefin product composition to be specifically approximated to the desired olefin product composition. The alternative is recycling of the unwanted olefins, which, on the one hand, increases the recirculation volume and hence the intensity of separation (electricity and steam requirements) of the MTO process. On the other hand, adjustment of the olefin product range solely by recycling the unwanted olefins is only possible with a high level of development intensity owing to the many chemical reactions during the MTO process and quickly leads to an increase in the amounts of by-products.

In a preferred embodiment, the process of the invention comprises the steps of:
A1) feeding a feed stream A comprising methanol into a dimethyl ether fixed bed reactor and catalytically converting methanol to dimethyl ether to obtain a product stream A1 comprising dimethyl ether, methanol, water vapor;
A2) mixing at least a portion of product stream A1 with at least one hydrocarbon recycle stream R comprising C₂-C₆ hydrocarbons and a water vapor stream to obtain a second feed stream A2;
B1) heating the second feed stream A2 in one or more heat exchangers to a temperature in the range from 430 to 500° C. and feeding it into an olefin fixed bed reactor, where the heating may also precede the mixing of individual substreams to give the feed stream A2 in step A2);
B2) catalytically converting feed stream A2 at a temperature in the range from 430 to 520° C. to a crude product gas stream B comprising ethylene, propylene, butenes, further C₂-C₆ hydrocarbons, C₇⁺ hydrocarbons, methanol and water vapor;
C1) cooling crude product gas stream B in one or more heat exchangers to a temperature in the range from 170 to 220° C. by heat exchange with feed stream A2;
C2) further cooling crude product gas stream B to a temperature in the range from 30 to 60° C. by contacting with at least one water-containing quench circulation stream K, with condensation of water and methanol, to obtain a water- and methanol-depleted hydrocarbon crude product gas stream C;
D) separating hydrocarbon crude product gas stream C into a propylene-comprising product-of-value stream, optionally an ethylene-comprising product-of-value stream, a butene-comprising product-of-value stream, at least one recycle stream comprising C₅-C₆ hydrocarbons, and at least one by-product stream comprising C₆⁺ hydrocarbons.

In step A1), a feed stream A comprising methanol is fed into a dimethyl ether fixed bed reactor and methanol is catalytically converted to dimethyl ether to obtain a product stream A1 comprising dimethyl ether, methanol and water vapor.

If, based on 100% by weight of the C₂-C₄ olefins obtained as products of value, 30% to 60% by weight of ethylene, 30% to 60% by weight of propylene and 0% to 30% by weight of butenes are to be obtained as products of value, the invention comprises, based on crude product stream B, conducting 20% to 40% of the propylene into the olefin metathesis reactor, and, based on the C₂-C₄ olefins present in the one or more recycle streams R and the product-of-value and by-product streams F1, F3, F4, H1, N and H3 or H4, recycling 0% to 10% of the ethylene, 30% to 60% of the propylene and 40% to 80% of the butenes into step B).

Alternatively, based on crude product stream B, 50% to 70% of the propylene can be conducted into the olefin metathesis reactor, and, based on the C₂-C₄ olefins present in the one or more recycle streams R and the product-of-value and by-product streams F1, F3, F4, H1, N and H3 or H4, 5% to 30% of the ethylene, 0% to 10% of the propylene and 40% to 85% of the butenes are recycled into step B).

100% by weight of products of value is based on the C₂-C₄ olefins present in streams F1, F3, F4, H1 and either H3 or H4 that are not recycled and are not fed into the metathesis reactor.

The dimethyl ether fixed bed reactor can be designed in various ways. In general, the feed gas stream A comprising methanol is heated up to a temperature of above 250° C. and fed into the fixed bed reactor. The catalyst used is generally gamma-alumina. The conversion temperature is between 25° and 450° C., and the pressure between 1 and 25 bar, for example 4 bar. The methanol conversion is generally 50% to 90%, preferably 65% to 85%, for example 75%.

In a step A2), at least a portion of said stream A1 is mixed with one or more hydrocarbon recycle streams R comprising C₂-C₆ hydrocarbons and a water vapor stream to obtain a feed gas stream A2. In general, this portion of stream A1 is at least 50% by weight and preferably up to 90% by weight. A further portion A1-2 of stream A1 of preferably at least 10% by weight can be fed directly to one or more trays of the olefin fixed bed reactor. This portion of stream A1 is generally cooled before being fed into the trays of the olefin fixed bed reactor, preferably to a temperature in the range from 30 to 60° C. This substream A1-2 is preferably fed into the reactor in liquid form.

The cooling by substream A1-2 can also be dispensed with if the olefin fixed bed reactor is operated and cooled isothermally. Isothermal operation can be effected, for example, in the manner described in WO 2017/102096 A1.

For instance, there may be heat transfer surfaces installed in the olefin fixed bed reactor that are operated, for example, with liquid salt or high-pressure steam as heat transfer medium. By virtue of the flow of the heat transfer medium through the heat transfer surfaces in the reactor, the heat of reaction that arises is removed from the reactor and hence this is operated isothermally. In order to establish the desired product distribution in product gas stream B, it is also possible here to feed a substream A1-2 to an intermediate stage of the olefin fixed bed reactor.

The at least one hydrocarbon recycle stream R that comes from the removal of the C₂-C₄ olefins in steps D) and optionally G) generally comprises C₂-C₆ hydrocarbons. According to the proportions in which ethylene, propylene and butenes are obtained as products of value, the at least one hydrocarbon recycle stream comprises C₂-C₄ hydrocarbons, generally in amounts of 40% to 90% by weight in total. The at least one hydrocarbon recycle stream prior to mixing is generally at a temperature in the range from 100 to 175° C., preferably in the range from 130 to 160° C.

The product stream A1 from the dimethyl ether fixed bed reactor is also mixed with a water vapor stream. The water vapor stream is generally at a temperature in the range from 100 to 200° C., preferably in the range from 100 to 150° C. The feed stream A2 thus obtained generally comprises 20% to 80% by weight, preferably 30% to 60% by weight, of water vapor. It generally further comprises 1% to 10% by weight of methanol, 0% to 15% by weight of ethanol, 1% to 20% by weight of dimethyl ether and 10% to 50% by weight of C₂-C₆ hydrocarbons.

In a step B1), the feed stream A2 is heated up in one or more heat exchangers to a temperature in the range from generally 430 to 500° C. and fed into an olefin fixed bed reactor. The heating may also take place prior to the mixing of individual substreams in step A2).

In general, feed stream A2 on feeding into the olefin fixed bed reactor is at a temperature in the range from 430 to 500° C., for example 470° C. Feed stream A2 can be heated to that temperature by heat exchange with the crude product gas stream B from the olefin fixed bed reactor, direct electrical heating, or heating via combustion of a separate fossil energy carrier.

This is followed in step B2) by catalytic conversion in the olefin fixed bed reactor to a product gas stream B comprising ethylene, propylene, butenes, further C₂-C₆ hydrocarbons, C₇⁺ hydrocarbons, methanol and water vapor. The conversion is generally effected over a zeolite catalyst, preferably over a catalyst based on a ZSM-5 zeolite. The reaction temperature is generally 430 to 500° C., preferably 460 to 480° C. The pressure is generally is 1.3 to 2.5 bar. The resultant crude product gas stream B2 is preferably of the following composition: 1% to 15% by weight of ethylene, 1% to 30% by weight of propylene, 35% to 80% by weight of water, 10% to 50% by weight of C₂-C₆ hydrocarbons, especially butenes, and saturated C₄ and C₅ hydrocarbons and C₆⁺ hydrocarbons, and 0.01% to 1.5% by weight of methanol and dimethyl ether.

The olefin fixed bed reactor generally takes the form of a tray reactor. The number of trays is preferably 4 to 6. In one embodiment, a total of up to 50% by weight of gas stream A is fed directly to one or more trays of the olefin fixed bed reactor, preferably all trays of the olefin fixed bed reactor. In a further embodiment, methanol is fed directly to one or more trays of the olefin fixed bed reactor, preferably all trays of the olefin fixed bed reactor.

The crude product gas stream B on exit from the reactor is at a temperature of generally 430 to 520° C., preferably 460 to 480° C.

In a preferred step C1), crude product gas stream B is cooled in one or more heat exchangers to a temperature in the range from 170 to 220° C. by heat exchange with feed gas stream A2. After this cooling step, the temperature of crude product gas stream B is generally 160 to 220° C., preferably 170 to 210° C., for example 190° C.

In a preferred step C2), crude product gas stream B is cooled further to a temperature in the range from 30 to 60° C. by contacting with one or more water-containing quench circulation streams, with condensation of water and methanol, to obtain a water- and methanol-depleted hydrocarbon crude product gas stream C. The hydrocarbon crude product gas stream C thus obtained comprises essentially ethylene, propylene, further C₂-C₆ hydrocarbons and C₇⁺ hydrocarbons.

In a step D), one or more product streams comprising C₂-C₄ olefins are separated from the hydrocarbon crude product gas stream C, and the at least one recycle stream R comprising C₂-C₆ hydrocarbons is obtained. The (overall) recycle stream R may comprise or be formed from multiple individual recycle streams R1, R2, R3 etc. In general, the overall recycle stream R comprising C₂-C₆ hydrocarbons comprises essentially, i.e. to an extent of >95% by weight, C₂-C₆ hydrocarbons.

In general, step D) comprises steps D1) to D7):
D1) compressing hydrocarbon crude product gas stream C to obtain a liquid hydrocarbon stream D11 comprising propylene and C₄, C₅ and C₆⁺ hydrocarbons, and an ethane-, ethene- and propylene-comprising gaseous hydrocarbon stream D12;
D2) separating water from the liquid hydrocarbon stream D11 by phase separation to obtain a liquid hydrocarbon stream D21;
D3) separating a propylene-comprising stream D31 from the liquid hydrocarbon stream
D21 to obtain a stream D32 comprising C₄, C₅ and C₆⁺ hydrocarbons; or separating a stream D31 comprising propylene and C₄ hydrocarbons to obtain a stream D32 comprising C₄, C₅ and C₆⁺ hydrocarbons;
D4) separating a by-product stream D41 comprising C₆⁺ hydrocarbons from the stream
D32 comprising C₄, C₅ and C₆⁺ hydrocarbons to obtain a stream D42 comprising C₄, C₅ and C₆ hydrocarbons; stream D41 optionally comprises aromatic C₆ hydrocarbons, and stream D42 aliphatic C₆ hydrocarbons;
D5) separating a propylene-comprising stream D51 from the ethane-, ethene- and propylene-comprising gaseous hydrocarbon stream D12 to obtain an ethane- and ethene-comprising stream D52;
D6) separating a stream D61 comprising butenes from the stream D42 comprising C₄, C₅ and C₆ hydrocarbons to obtain a stream D62 comprising C₅ and C₆ hydrocarbons; and or separating a propylene-comprising stream D63 from stream D31 to obtain a stream D64 comprising butenes;
D7) obtaining at least one recycle stream R from one or more of the streams selected from stream D42 comprising C₄, C₅ and C₆ hydrocarbons, stream D62 comprising C₅ and C₆ hydrocarbons, the propylene-comprising stream D31, the propylene-comprising stream D51, the propylene-comprising stream D63, stream D61 comprising butenes, stream D64 comprising butenes, and the ethane- and ethene-comprising stream D52.

Steps D3), D4), D5) and D6) are conducted in standard distillation apparatuses. Useful distillation apparatuses in principle include the apparatuses known to the person skilled in the art for such separation tasks. As well as the actual column body with internals, the distillation column, as usual, also comprises a top condenser and a reboiler. The column body may be equipped, for example, with structured packings, random packings or trays. The distillation apparatuses may be designed and operated by the common knowledge of the person skilled in the art.

In step E), a portion of the C₂-C₄ olefins and at least a portion of the C₅-C₆ hydrocarbons are recycled into step B) as one or more hydrocarbon recycle streams R.

In step F), at least one ethylene-comprising product-of-value stream F1, at least one propylene-comprising product-of-value stream F2, and possibly one or more product-of-value streams F3 comprising butenes are obtained.

Propylene can be obtained as product of value from streams D31 or D63 and D51. Ethylene can be obtained as product of value from stream D52. Butenes can be obtained from stream D61 and/or D64.

The recycle stream(s) R may be obtained from one or more of the above-described streams D31, D42, D51, D52, D61, D62, D63 and D64.

In step G), at least a portion of the propylene-comprising product-of-value stream F2 is fed into an olefin metathesis reactor and propylene is converted to a product stream G comprising ethylene and butenes.

The conversion is generally effected in the gas phase. Suitable metathesis catalysts are, for example, tungsten oxide on silicon dioxide (WO₃/SiO₂), cobalt molybdate on aluminum oxide (CoO—MO₃/Al₂O₃) and rhenium oxide on aluminum oxide (Re₂O₇/Al₂O₃). The reaction may, as described in U.S. Ser. No. 11/136,278, take place within the temperature range from 50 to 650° C. and the pressure range from 0 to 40 bar (g).

The metathesis reaction of propylene involves a disproportionation of propylene to ethylene and 2-butene. This is an equilibrium reaction. The metathesis catalyst may additionally comprise an isomerization catalyst, for example magnesium oxide (MgO), which catalyzes the isomerization of 2-butene to 1-butene.

A product stream G comprising ethylene, propylene and butenes is obtained.

In step H), an ethylene-comprising product-of-value stream H1 and a product-of-value stream H3 comprising butenes are separated from the product stream G.

The separation H) generally comprises
H1) the separating of an ethylene-comprising product-of-value stream H1 from product stream G in an ethylene separation column as a tops discharge stream to obtain a bottom draw stream comprising propylene and butenes;
H2) the separating of a propylene-comprising stream H2 from the bottom draw stream comprising propylene and butenes in a propylene separation column as tops discharge stream to obtain a bottom draw stream comprising butenes. The latter can be obtained directly as a product-of-value stream H3 or optionally purified further by distillation.

The propylene-comprising stream H2 is preferably recycled into the metathesis reactor.

In a step I), at least a portion of the product-of-value stream H3 comprising butenes is optionally recycled into step B).

In one embodiment of the invention, 50% to 100% of the butenes present in the product-of-value stream H3 is recycled into step B). Alternatively, a portion of streams D61 and/or D64 that corresponds to the same amount of butene can be recycled.

In step K), a by-product stream comprising C₆⁺ hydrocarbons is discharged from the process. This may be stream D41.

The invention is elucidated in detail by the examples below.

### EXAMPLES

Cases 1-3 below were simulated by computation. The results obtained were the compositions and relative mass flow rates of the product-of-value streams, by-product streams and recycle streams. The proportions of olefins into the recycling and metathesis were varied. The figures are weight ratios.

In order to ascertain the preferred ranges, catalytic experiments were conducted on a laboratory scale, on the basis of which the expected conversions and mass flow rates in an industrial process with a preliminary reactor, tray reactors and separation section were calculated. Analysis of this system led to cases 1-3 that are reported in table 1. Tables 2-4 show the relative mass flow rates and total composition of the main and by-product streams, and also the recycle streams. As can be seen in the tables, a metathesis can reduce the number of by-products and the volume of the return stream, and hence the energy efficiency and mass efficiency of the process can be improved. At the same time, a broader range of desired olefins is obtained than in the prior art.

TABLE 1

| | Case 1 | | Case 2 | | Case 3 | |
|---|---|---|---|---|---|---|
| | Min | Max | Min | Max | Min | Max |
| Recycled portion | 0% | 0% | 0% | 10% | 10% | 30% |
| of C₂H₄ from the | | | | | | |
| crude product | | | | | | |
| stream^{[1]} | | | | | | |
| Recycled portion | 60% | 100% | 20% | 40% | 0% | 10% |
| of C₃H₆ from the | | | | | | |
| crude product | | | | | | |
| stream[^{1]} | | | | | | |
| Portion of C₃H₆ | | | 20% | 40% | 50% | 70% |
| from the crude | | | | | | |
| product | | | | | | |
| stream^{[1]} | | | | | | |
| fed into the | | | | | | |
| metathesis | | | | | | |
| reactor | | | | | | |
| Recycled portion | 40% | 70% | 30% | 100% | 30% | 100% |
| of C₄H₈ from the | | | | | | |
| crude product | | | | | | |
| stream^{[1]} | | | | | | |
| Recycled portion | 0% | 0% | 0% | 10% | 5% | 30% |
| of C₂H₄ relative | | | | | | |
| to C₂H₄ in the | | | | | | |
| recycling, the | | | | | | |
| products of | | | | | | |
| value and by- | | | | | | |
| products^{[2]} | | | | | | |
| Recycled portion | 60% | 100% | 30% | 60% | 0% | 10% |
| of C₃H₆ relative | | | | | | |
| to C₃H₆ in the | | | | | | |
| recycling, the | | | | | | |
| products of | | | | | | |
| value and by- | | | | | | |
| products^{[2]} | | | | | | |
| Recycled portion | 40% | 70% | 40% | 80% | 40% | 85% |
| of C₄H₈ relative | | | | | | |
| to C₄H₈ in the | | | | | | |
| recycling, the | | | | | | |
| products of | | | | | | |
| value and by- | | | | | | |
| products^{[2]} | | | | | | |

^{[1]}After separation in step D)
^{[2]}After separation in step D) and step H)

TABLE 2

| | Case 1: No metathesis unit | | |
|---|---|---|---|
| | Products of | | |
| | value | By-products | Recycling |
| Relative mass flow | 1.00 | 1.72 | 3.08 |
| rate | | | |
| Proportions by mass | | | |
| MeOH | — | — | — |
| DME | — | 0.01 | 0.01 |
| H2O | — | — | — |
| H2 | — | 0.00 | — |
| CO2 | — | 0.00 | — |
| CH4 | — | 0.02 | — |
| C2H4 | 0.35 | — | — |
| C3H6 | 0.38 | — | 0.43 |
| C4H8 | 0.27 | 0.00 | 0.11 |
| C5H10 | — | 0.02 | 0.01 |
| C6+ olefins | — | 0.01 | 0.01 |
| C2H6 | — | 0.07 | — |
| C3H8 | — | 0.11 | 0.21 |
| C4H10 | — | 0.19 | 0.13 |
| C5H12 | — | 0.11 | 0.06 |
| C6+ paraffins | — | 0.06 | 0.03 |
| Aromatics | — | 0.41 | — |
| N2 | — | — | — |

TABLE 3

| | Case 2: No recycled C₂H₄ | | |
|---|---|---|---|
| | Products of | | |
| | value | By-products | Recycling |
| Relative mass flow | 1.00 | 0.88 | 1.85 |
| rate | | | |
| Proportions by mass | | | |
| MeOH | — | — | — |
| DME | — | 0.04 | — |
| H2O | — | — | — |
| H2 | — | 0.00 | — |
| CO2 | — | 0.00 | — |
| CH4 | — | 0.02 | — |
| C2H4 | 0.36 | — | — |
| C3H6 | 0.41 | — | 0.18 |
| C4H8 | 0.23 | 0.00 | 0.21 |
| C5H10 | — | 0.02 | 0.01 |
| C6+ olefins | — | 0.02 | 0.01 |
| C2H6 | — | 0.16 | — |
| C3H8 | — | 0.11 | 0.02 |
| C4H10 | — | 0.01 | 0.49 |
| C5H12 | — | 0.12 | 0.06 |
| C6+ paraffins | — | 0.07 | 0.03 |
| Aromatics | — | 0.42 | — |
| N2 | — | — | — |

TABLE 4

| | Case 3: no recycled C₃H₆ | | |
|---|---|---|---|
| | Products of | | |
| | value | By-products | Recycling |
| Relative mass flow | 1.00 | 0.88 | 0.85 |
| rate | | | |
| Proportions by mass | | | |
| MeOH | — | — | — |
| DME | — | 0.04 | — |
| H2O | — | — | — |
| H2 | — | 0.00 | — |
| CO2 | — | 0.00 | — |
| CH4 | — | 0.01 | — |
| C2H4 | 0.37 | — | — |
| C3H6 | 0.39 | — | — |
| C4H8 | 0.24 | 0.01 | 0.62 |
| C5H10 | — | 0.02 | 0.02 |
| C6+ olefins | — | 0.01 | 0.01 |
| C2H6 | — | 0.05 | 0.01 |
| C3H8 | — | 0.09 | |
| C4H10 | — | 0.21 | 0.12 |
| C5H12 | — | 0.11 | 0.11 |
| C6+ paraffins | — | 0.06 | 0.06 |
| Aromatics | — | 0.38 | — |
| N2 | — | — | — |

1.-4. (canceled)

5. A process for preparing C₂-C₄ olefins from methanol, comprising the steps of:
A) feeding a feed stream A comprising methanol into a dimethyl ether fixed bed reactor and catalytically converting methanol to dimethyl ether to obtain a product stream A1 comprising dimethyl ether, methanol and water vapor;
B) mixing stream A1 with one or more hydrocarbon recycle streams R comprising C₂-C₆ hydrocarbons and catalytically converting it in an olefin fixed bed reactor to a crude product stream B comprising C₂-C₄ olefins, C₅-C₆ hydrocarbon and C₆⁺ hydrocarbons;
C) cooling crude product stream B to obtain a hydrocarbon crude product stream C;
D) separating hydrocarbon crude product stream C into at least one ethylene-comprising stream, at least one propylene-comprising stream, at least one stream comprising butenes, at least one stream comprising C₅-C₆ hydrocarbons, and at least one by-product stream N comprising C₆⁺ hydrocarbons;
E) recycling a portion of the C₂-C₄ olefins selected from ethylene, propylene and butenes that are obtained in step D), and at least a portion of the C₅-C₆ hydrocarbons that are obtained in step D) as one or more hydrocarbon recycle streams R into step B);
F) obtaining at least one ethylene-comprising product-of-value stream F1, at least one propylene-comprising product stream F2 and optionally one or more product-of-value streams F3 comprising butenes from the streams obtained in step D);
G) feeding at least a portion of the propylene-comprising product stream F2 into an olefin metathesis reactor and converting propylene to a product stream G comprising ethylene and butenes, leaving a substream F4 of stream F2 as the product-of-value stream;
H) separating an ethylene-comprising product-of-value stream H1 and a product-of-value stream H3 comprising butenes from the product stream G;
I) optionally recycling at least a portion of the butenes present in product-of-value stream H3 into step B), leaving a substream H4 as product-of-value stream; and
J) discharging the at least one by-product stream N comprising C₆⁺ hydrocarbons;
wherein
based on 100% by weight of the C₂-C₄ olefins present as products of value in streams F1, F3, F4, H1 and H3 or H4, 30% to 60% by weight of ethylene, 30% to 60% by weight of propylene and 0% to 30% by weight of butenes are present and are recovered as products of value, and, based on C₂-C₄ olefins present in the one or more recycle streams R and the product-of-value and by-product streams F1, F3, F4, H1, N and H3 or H4, 0% to 10% of the ethylene, 30% to 60% of the propylene and 40% to 80% of the butenes are recycled into step B), and, based on crude product stream B, 20% to 40% of the propylene is conducted into the olefin metathesis reactor, or, based on the C₂-C₄ olefins present in the one or more recycle streams R and the product-of-value and by-product streams F1, F3, F4, H1, N and H3 or H4, 5% to 30% of the ethylene, 0% to 10% of the propylene and 40% to 85% of the butenes are recycled into step B), and, based on crude product stream B, 50% to 70% of the propylene is conducted into the olefin metathesis reactor.

6. The process according to claim 5, wherein steps A) to D) comprise the following steps A1), A2), B1), B2), C1), C2) and D):
A1) feeding a feed stream A comprising methanol into a dimethyl ether fixed bed reactor and catalytically converting methanol to dimethyl ether to obtain a product stream A1 comprising dimethyl ether, methanol and water vapor;
A2) mixing at least a portion of product stream A1 with at least one hydrocarbon recycle stream R comprising C₂-C₆ hydrocarbons and a water vapor stream to obtain a second feed stream A2;
B1) heating the second feed stream A2 in one or more heat exchangers to a temperature in the range from 430 to 500° C. and feeding it into an olefin fixed bed reactor, where the heating may also precede the mixing of individual substreams to give the feed stream A2 in step A2);
B2) converting, catalytically, feed stream A2 at a temperature in the range from 430 to 520° C. to a crude product gas stream B comprising ethylene, propylene, butenes, further C₂-C₆ hydrocarbons, C₆⁺ hydrocarbons, methanol and water vapor;
C1) cooling crude product gas stream B in one or more heat exchangers to a temperature in the range from 170 to 220° C. by heat exchange with feed stream A2;
C2) cooling crude product gas stream B to a temperature in the range from 30 to 60° C. by contacting with at least one water-containing quench circulation stream K, with condensation of water and methanol, to obtain a water- and methanol-depleted hydrocarbon crude product gas stream C2; and
D) separating hydrocarbon crude product stream C into an ethylene-comprising stream D1, a propylene-comprising stream D2, a stream D3 comprising butenes, at least one stream D4 comprising C₅-C₆ hydrocarbons, and at least one by-product stream N comprising C₆⁺ hydrocarbons.

7. The process according to claim 5, wherein step D) comprises steps D1) to D7):
D1) compressing hydrocarbon crude product gas stream C to obtain a liquid hydrocarbon stream D11 comprising propylene and C₄, C₅ and C₆⁺ hydrocarbons, and an ethane-, ethene- and propylene-comprising gaseous hydrocarbon stream D12;
D2) separating water from the liquid hydrocarbon stream D11 by phase separation to obtain a liquid hydrocarbon stream D21;
D3) separating a propylene-comprising stream D31 from the liquid hydrocarbon stream D21 to obtain a stream D32 comprising C₄, C₅ and C₆⁺ hydrocarbons; or separating a stream D31 comprising propylene and C₄ hydrocarbons to obtain a stream D32 comprising C₄, C₅ and C₆⁺ hydrocarbons;
D4) separating a by-product stream D41 comprising C₆⁺ hydrocarbons from the stream D32 comprising C₄, C₅ and C₆⁺ hydrocarbons to obtain a stream D42 comprising C₄, C₅ and C₆ hydrocarbons; stream D41 optionally comprises aromatic C₆ hydrocarbons, and stream D42 aliphatic C₆ hydrocarbons;
D5) separating a propylene-comprising stream D51 from the ethane-, ethene- and propylene-comprising gaseous hydrocarbon stream D12 to obtain an ethane- and ethene-comprising stream D52;
D6) separating a stream D61 comprising butenes from the stream D42 comprising C₄, C₅ and C₆ hydrocarbons to obtain a stream D62 comprising C₅ and C₆ hydrocarbons; and or separating a propylene-comprising stream D63 from stream D31 to obtain a stream D64 comprising butenes; and
D7) obtaining at least one recycle stream R from one or more of the streams selected from stream D42 comprising C₄, C₅ and C₆ hydrocarbons, stream D62 comprising C₅ and C₆ hydrocarbons, the propylene-comprising stream D31, the propylene-comprising stream D51, the propylene-comprising stream D63, stream D61 comprising butenes, stream D64 comprising butenes, and the ethane- and ethene-comprising stream D52.

8. The process according to claim 5, wherein in step I) 50% to 100% of the butenes present in product-of-value stream H3 is recycled into step B).
